(19) United States
(12) Patent Application Publication
(10) Pub. No.: US 2021/0402001 A1
(43) Pub. Date: Dec. 30, 2021

(54) Zinc Porters, and their Monoclonal Antibody Conjugates, for the Prevention and Treatment of COVID-19 (SARS-CoV-2), Other Infections, and Cancers

(71) Applicant: David I. Cohen, Mount Vernon, NY (US)

(72) Inventor: David I. Cohen, Mount Vernon, NY (US)

(21) Appl. No.: 17/189,918

(22) Filed: Mar. 2, 2021

Related U.S. Application Data
(60) Provisional application No. 63/046,225, filed on Jun. 30, 2020.

(51) Int. Cl. A61K 47/68; A61P 31/14; A61K 47/52; A61K 39/39; A61P 31/16; A61P 35/00; A61K 38/17

(52) U.S. Cl. CPC A61K 47/6841; A61P 31/14; A61K 47/6811; A61K 47/52; A61K 38/1729; A61P 31/16; A61P 35/00; A61K 47/6855; A61K 39/39

(57) ABSTRACT

The present invention describes how a MAb specific to a viral or cancer antigen can be linked to a novel Zn porting peptide that is engineered to load ionic Zn absent requirement for biological catalysis, carry Zn stably through the circulation, release payload proximal to diseased cells, deliver its Zn cargo intracellularly due to ionophore activity, and release Zn intracellularly. As specifically designed for COVID-19, the payload can be released by furin cleavage, and the Zn released intracellularly by cleavage at a 3CL major COVID protease site replacing sequences naturally found in alpha defensin-5, as one example, or by many other variations encompassing this design. The peptide's entry and intracellular Zn release can be further facilitated by the insertion of an arginine-lysine rich membrane translocation sequence at its amino or carboxyl terminus. The design provides a novel unifying strategy for preventing and treating COVID-19 (SARS-CoV-2) infection, other coronaviral infections, influenza infections, and many cancers.

### CROSS RELATED APPLICATIONS

This application claims the benefit of U.S. Provisional Patent Application No. 63/046,225, Filed 30 Jun. 2020, the disclosure of which is incorporated herein by reference.

### FIELD OF THE INVENTION

The present invention relates to controlling COVID-19 infections and their progression to SARS-CoV-2 morbidities and mortalities, through the targeted, intracellular delivery of zinc (Zn) at the sites of viral infections. The COVID-19-specific Zn porters can be rapidly manufactured at scale. Their specificity can be further enhanced, and therapeutic thresholds achieved more readily, by conjugation with monoclonal antibody (MAb) drugs directed at COVID-19. The Zn porters offer durable treatment against coronavirus infections because Zn inhibits all coronaviral RNA polymerases characterized to date, and their conjugates also suppress vaccine escape variants when their MAb component recognizes conserved coronaviral antigens. The invention has broad applications for other infectious diseases such as influenza, and cancers.

### BACKGROUND OF THE INVENTION

A general strategy against the current COVID-19 (SARS-CoV-2) pandemic, and any subsequent waves arising from antigenic variants, has been assembled. The strategies are built by synthetically evolving Zn-porting ionophores, engineered with proteolytic sites so as to release intracellular Zn in the context of COVID-19 infection. The Zn carriers are then administered alone, or coupled to anti-COVID MAb. Through MAb coupling, specificity of drug delivery can be enhanced, and thereby the dosing required for efficacy lowered. A low dose requirement in turn renders the mass scale production required to control pandemics more feasible. By delivering Zn intracellularly in a regulated fashion at sites of infection, the Zn porters attack both COVID-19 replication directly, because Zn is a potent inhibitor of COVID-19 and other coronaviral RNA polymerases, and the sequelae of SARS-CoV-2 disease attributable to Zn dysregulation and intracellular Zn depletion, specifically: 1) development of bacterial pneumonias, most prevalent in the elderly; 2) thrombotic events; and 3) defective antibody maturation resulting in persistent, explosive spread of COVID-19. Because Zn attacks many of the causes of severe SARS-CoV-2 disease, it can reposition for success MAb therapeutics that have already failed as stand alone agents in advanced disease. The invention is a realization of retrospective clinical studies suggesting that Zn (in combination therapy) could have a mitigating effect on COVID-19 infections.

The current COVID-19 pandemic requires urgent interventions with rapidly-deployed agents. Johns Hopkins reports as of December 2020, more than 81 million COVID-19 infections world-wide, and almost 1.8 million SARS-CoV-2 deaths, both surely underestimates. Currently, COVID infections are rising, with the most new cases in USA, United Kingdom. Russia, Brazil, and India. While distancing, wearing masks, and testing measures flattened the first wave of COVID-19 infections in New York State, the original US epicenter, the emergence of a second wave, even in New York State, means these and other public health measures overall are inadequate to end the COVID-19 pandemic. It remains to be determined whether the many COVID-19 vaccines can turn the tide against COVID-19 spread, but it is clear the vaccines cannot prevent deaths in those that have already become symptomatically infected. COVID-19 hospitalizations on average cost approximately $50,000 per individual, translating into a burden from SARS-CoV-2 hospitalizations projecting to exceed $100 billion worldwide in 2021.

Prior use of siderophores by Nolan et al., where the cargo is an antibiotic, a fluorophore, or biotin, does not disclose a Zn-porting therapeutic provided in application U520150105337, or its coupling to a monoclonal antibody conjugates as provided in the present invention. Conjugates in Nolan et al. are further used for the treatment of bacterial infections and other diseases, but not viral infections or cancer as described herein. Shoulders et al. describes methods for sequestering metal ions, including Zn. Shoulders does not describe methods for delivering Zn as a therapeutic, or methods by which Zn can be delivered intracellularly, see application US20190210005. In addition, Nagourney et al. claims a method for identifying metabolite signatures of various diseases, including COVID 19, that can be used as an assay for diagnosis and treatment. Noteworthy is the lack of any reference to the use of Zn or incorporation into a method relevant to Zn dysregulation in COVID-19 disease with no claims directed to treatment, see application U520200386766.

### SUMMARY OF THE INVENTION

A conjugate for treating and preventing SARS-CoV-2 disease is described that couples a MAb specific to a COVID-19 Spike, or other externally-exposed, protein, to a novel Zn porting peptide. The porting peptide is engineered to load ionic Zn absent requirement for biological catalysis, carry Zn stably through the circulation, deliver its Zn cargo intracellularly due to ionophore activity, and release Zn once inside an infected cell owing to COVID-19 specific protease cleavage sites designed into the peptide. The peptide's entry and intracellular release of Zn can be further facilitated by the insertion of an arginine-lysine rich membrane translocation sequence at its amino or carboxyl terminus.

### DESCRIPTION OF THE DRAWINGS

FIG. 1. Cargo Carrying an alpha defensin chelating Zn, with the alpha defensin modified to contain COVID-19 protease Cleavage Sites.

FIG. 2. Anti-COVID-19 Potency of DF-MCP.

FIG. 3. Potency of Zn in combination with Ionophore in Inhibiting COVID-19 Replication.

FIG. 4. Anti Covid-19 Activity of DF-PLP.

FIG. 5. Schematic of a Precisely targeted Zn Payload through coupling a COVID-specific MAb to a COVID-activated Zn Porter/ionophore.

FIG. 6. Diagram of a synthetically-evolved alpha Defensin targeted to release Zn inside of influenza-infected cells.

FIG. 7. Schematic of a Precisely targeted Zinc Payload through, coupling an Influenza-specific MAb to a Zinc Porter/ionophore.

FIG. 8. Schematic of a Zn Payload Precisely targeted to a cancer, through coupling to a MAb specific to a cancer (Neo) Antigen.

### DETAILED DESCRIPTION

Finely-targeted, improved Zn porting is achieved by linking a Zn-coordinating peptide to a MAb directed against COVID-19 external antigens. For MAb that already deliver anti-COVID-19 neutralizing activity, such as those directed against COVID-19 Spike (S) proteins that block viral attachment to a cell, Zn cargo can enhance anti-COVID activities of the MAb through its synergistic activity to inhibit viral replication intracellularly. Further, this synergy can target the MAb-Zn conjugate to treat severe COVID-19 infections after disease has become refractory to MAb. because its Zn component can reverse SARS pathologies arising from Zn dysregulation. Owing to the exquisite sensitivity of SARS-CoV-2 to Zn, a MAb-targeted conjugate can achieve therapeutic thresholds at substantially lower dosing, facilitating manufacture of enough MAb conjugates to treat mass populations in the middle of a pandemic. One MAb can target multiple Zn payloads linked in tandem to the MAb heavy chains, further enhancing the potency of the conjugate.

Conjugation of the Zn porter to an MAb recognizing a conserved coronavirus antigen, whether that epitope is neutralizing or not, generates a pandemic-neutralizing drug capable of treating and preventing subsequent COVID-19 waves initiated by viral antigenic variants. The Zn component of the conjugate, with its safely regulated Zn release upon COVID infection, makes for a universal blockade against coronaviral replication. Such conjugates can suppress emergence of COVID variants that otherwise could escape current vaccines. When the MAb is itself neutralizing, synergistic activities are achieved that can promote the efficacies and extend the duration of protection of pre-exposure (PREP) and post-exposure (PEP) prophylaxis of both drugs. These conjugates then have quadruple advantages of their activities for treating and preventing COVID, lowering dosage requirements, suppressing emergence of resistance variants so that herd immunity through mass vaccination with current vaccines becomes more achievable, and preparing for any future COVID pandemics.

Accordingly, the treatment methods of the present invention may involve administration of the Zn porter either by itself, or when conjugated with Mab. The drugs can be delivered alone, or with a suitable pharmaceutical composition comprising a pharmaceutically acceptable carrier or delivery system, such as an adjuvant used in subcutaneous or intramuscular therapy.

Generally, these drugs are prepared as injectables, in the form of aqueous solutions or suspensions. Solid forms that are dissolved or suspended prior to use may also be formulated. Oral forms of the Zn porter can be administered to safely attack latent intestinal COVID reservoirs, to prevent the emergence of resistant strains from the gut microbiota. Pharmaceutical carriers, diluents and excipients are generally added that are compatible with the active ingredients and acceptable for pharmaceutical use. Examples of such carriers include, but are not limited to, water, saline solutions, dextrose, or glycerol. Combinations of carriers may also be used. These compositions may be sterilized by conventional, well known sterilization techniques including sterile filtration. The resulting solutions may be packaged for use as is, or the aqueous solutions may be lyophilized, the lyophilized preparation being combined with sterile water before administration. Compositions may further incorporate additional substances to stabilize pH, or to function as adjuvants, wetting agents, or emulsifying agents, which can serve to improve the effectiveness of the delivery system.

Zn in a PREP formulation lies safely sequestered within the conjugate, waiting to be released when COVID-19 exposure triggers the synthesis of virally-regulated proteases. This feature mitigates against extracellular Zn toxicities, that are a source for clotting abnormalities such as thrombotic strokes and heart attacks, which were reported to be 10 times more frequent than normal in young people at the onset of the COVID-19 epidemic in New York City; and loss of smell (anosmia), which is a common COVID-19 diagnostic feature in otherwise asymptomatic individuals. Further, the ionophore activity of the porter assures that Zn is released intracellularly, where it attacks COVID-19, and not into the circulation where it causes systematic toxicities, that are enhanced when individuals self-medicate with dietary zinc supplements.

Hypercoagulability, as observed in advancing SARS disease, is a major consequence of excess intravascular Zn, because blood platelets, a principal extracellular Zn storage reservoir, initiate blood clotting through Zn release. Platelets become hyperactive in healthy individuals after a single oral dose of 220 mg Zn sulfate, the amount in many dietary supplements. Anosmia, an early indicator of COVID-19 infection in otherwise healthy, young individuals, can result from nasal Zn overload. Iatrogenic acute anosmia due to excess intranasal Zn was caused when drug companies attempted to replace oral Zn supplements with inhaled Zn as a treatment for the common cold. Intranasal Zn sulfate, trialed during the polio epidemic as a chemical blockade to poliovirus infection, resulted instead in profound acute anosmia, some of which was permanent.

A Zn porter peptide that releases its Zn payload intracellularly, and only during COVID-19 infections, is designed (“synthetically evolved”) from the natural sequences of Zn fingers, preferably alpha defensins because these carry intrinsic ionophore activity, through the insertion of a COVID-19 sensitive protease site between the two halves of the Zn finger's cysteine-rich sequences (CRS). SARS-CoV-2 has two viral-specific proteases, SARS-2 3CL main viral protease (3CLpro, Main COVID Protease, (MCP)), and SARS-2 papain-like protease (PLP) with distinct recognition sequences. Together, the paired CRS function as a Zn finger to grab Zn. Insertion of a COVID-sensitive protease site configures the peptide for COVID-specific cleavage that separates the Zn finger into two halves, thereby proteolytically releasing Zn. The ionophore activity of the defensin moves Zn into the cell, where COVID proteases are expressed during viral maturation. Cleavage of the peptide at its COVID proteolytic site delivers Zn intracellularly. Addition of an arginine-lysine rich membrane translocation sequence can further instruct the peptide into the lysosome to promote Zn release.

While any Zn-coordinating CRS or cysteine-histidine rich sequence is a process of this invention, alpha defensins (numbering 6 in humans) additionally have intrinsic ionophore, antibacterial, and antiviral activities that improve their characteristics. Alpha defensins appear to function in organisms as disparate as plants and human gut (Human Defensin 5, HDEFA5) as natural mediators of Zn homeostasis. HDEFA5 carries two staphylococcal peptidase 1 sites in its loop region between its Zn finger halves that could naturally release Zn at a pulmonary infection caused by staphylococcus. Other bacterial or viral protease sites are naturally located between the CRS of other alpha defensins, rendering them suitable templates for design of COVID-specific Zn porters, through replacement of these sequences with COVID-specific protease sites.

HDEFA5 is a cysteine-rich peptide (32 amino acid) that in its reduced state carries five trypsin cleavage sites, so it is rapidly degraded by gut trypsin. Oxidization of HDEFA5 opens up a canonical Zn finger that chelates Zn with a picomolar Zn dissociation constant. Chelation of oxidized HDEFA5 with free Zn blocks four of its five trypsin sites, leaving accessible only the poorest fitting site, thereby enabling HDEFA5 as a Zn carrier/reservoir that can survive in the trypsin-rich gut microenvironment.

Synthetically-evolved alpha defensin 5 (DF-COV) is easily loaded with Zn absent a requirement for biological catalysts. This confers upon DF-COV superior manufacturing characteristics. The protease site separating the two halves of the synthetic defensin can be a recognition sequence for either of the two COVID-19 encoded proteases, Chymotrypsin-like Major Covid Protease (3CLMCP, nsp5, making DF-MCP) or Papain-like Covid Protease (PLCP, nsp3, making DF-PLP), a caspase 8 recognition site selected because COVID infection triggers caspase 8-dependent apoptosis, or other proteases induced in the infected intracellular microenvironment. Enhanced Zn release could be attained with tandem SARS-CoV-2 protease cleavage sites in the region between the Zn fingers, or even placing both COVID-19 protease cleavage sites in this region.

Synthetically-evolved defensins specific to COVID-19 proteases (DF-MCP and DF-PLP) are engineered to alleviate toxicity from uncontrolled extracellular Zn release (“Zn storm”) by targeting free Zn intracellularly to sites of COVID-19 infection, whereas natural alpha defensins might not unload their Zn cargo at all, or could unload in the systemic circulation. Once COVID infection is extinguished, no further Zn is released from its carrier peptide, because there are no cellular proteases closely enough related to SARS-CoV-2 proteases, 3CLMCP and PLCP, to efficiently cleave the modified defensins. The process is autoregulatory. Autoregulation also follows from free Zn inhibiting 3CLMCP in addition to viral replication complexes, while Zn complexes, such as DF-PLP inhibit PLCP protease. Because Zn is released from the carrier intracellularly and is autoregulated to cease once COVID, and the proteases it encodes, no longer are produced, toxicities from excess extracellular Zn, such as severe anemia, excessive clotting, and strokes are mitigated.

TABLE 1
Description of Zn porter peptides showing sequence and peptide name.

| SEQ ID | | Protein |
|---|---|---|
| NO | Peptide | Name |
| SEQ ID | ATCYCRHGRCVKLQSADAVCEISGRLYRLCCR | DF-MCP-1 |
| No. 1 | | |
| SEQ ID | ATCYCRHGRCELNGGAVTRVCEISGRLYRLCCR | DF-PLP-1 |
| No. 2 | | |
| SEQ ID | ATCYCRHGRCVKLQSADAVCEISGRLYRLCCRRKKRRQRRR | DF-MCP-L |
| No. 3 | | |
| SEQ ID | ATCYCRHGRCELNGGAVTRVCEISGRLYRLCCRRKKRRQRRR | DF-PLP-L |
| No. 4 | | |
| SEQ ID | TNSPRRARSVAATCYCRHGRCVKLQSADAVCEISGRLYRLCCR | FU-DFMCP |
| No. 5 | | |
| SEQ ID | TNSPRRARSVAATCYCRHGRCELNGGAVTRVCEISGRLYRLCCR | FU-DFPLP |
| No. 6 | | |
| SEQ ID | PSKRSFIEATCYCRHGRCVKLQSADAVCEISGRLYRLCCR | TMP-DFMCP |
| No. 7 | | |
| SEQ ID | PSKRSFIEATCYCRHGRCELNGGAVTRVCEISGRLYRLCCR | TMP-DFPLP |
| No. 8 | | |
| SEQ ID | LFGFVGATCYCRHGRCVKLQSADAVCEISGRLYRLCCR | CB-DFMCP |
| No. 9 | | |
| SEQ ID | LFGFVGATCYCRHGRCELNGGAVTRVCEISGRLYRLCCR | CB-DFPLP |
| No. 10 | | |
| SEQ ID | DETDSPTVATCYCRHGRCVKLQSADAVCEISGRLYRLCCR | C8-DFMCP |
| No. 11 | | |
| SEQ ID | DETDSPTVATCYCRHGRCELNGGAVTRVCEISGRLYRLCCR | C8-DFPLP |
| No. 12 | | |
| SEQ ID | ATCYCRTGRCATRESLSGVCEISGRLYRLCCR | ADF5 |
| No. 13 | | |
| SEQ ID | ATCYCRTGRCATRESLSGVCEISGRLYRLCCR | DFL |
| No. 14 | RKKRRQRRR | |
| SEQ ID | ATCYCRTGRCDEVDDEVDVCEISGRLYRLCCR | DF-C3 |
| No. 15 | | |
| SEQ ID | QRKRKKRGATCYCRTGRCATRESLSGVCEISGRLYRLCCR | FLUC-DFL |
| No. 16 | RKKRRQRRR | |
| SEQ ID | PSIQYRGLFATCYCRTGRCATRESLSGVCEISGRLYRLCCR | FLUH1N1-DFL |
| No. 17 | RKKRRQRRR | |
| SEQ ID | PEKQTRGLFATCYCRTGRCATRESLSGVCEISGRLYRLCCR | FLUH3N2- |
| No. 18 | RKKRRQRRR | DFL |
| SEQ ID | PENPKTRGLFATCYCRTGRCATRESLSGVCEISGRLYRLCCR | FLUH7N7- |
| No. 19 | RKKRRQRRR | DFL |
| SEQ ID | MPLGIRMATCYCRTGRCATRESLSGVCEISGRLYRLCCR | MMP-DFL |
| No. 20 | RKKRRQRRR | |
| SEQ ID | ACYCRIPACIAGERRYGTCIYQGRLWAFCC | DFA1m |
| No. 21 | | |

Conjugation of the Zn porters to MAb specific for surface-expressed COVID determinants, particularly Spike (S) protein, offers dual advantages of superior targeting and synergistic activities against COVID, reducing the amount of drug required to achieve therapeutic thresholds. Efficient manufacture, as well as tightly controlled payload release, is attained by inserting a cleavage site recognized by an extracellular protease induced by COVID-19 as a genetic linker between the MAb and the Zn porter. An ideal candidate linker is COVID-19's unique furin site, (FU-Peptide) because COVID-19 infection employs surface-active furin protease to cleave budding S into 51 and S2 components. A further S2 cleavage, primarily mediated by the serine protease TMPRSS2 exposes the S2-cell fusion domain, rendering this sequence (TMP-Peptide) another preferred linker. An anti-S MAb targets conjugate right to the sites of both of these protease activities. The standard antibody drug conjugation linker cathepsin B (CB-Peptide) is also suitable, particularly since S protein is cleavable by cathepsins. Caspase 8 is the predominant apoptotic protease induced by COVID, and it further accumulates in the bloodstream in ongoing inflammation. A Caspase 8 protease site is a fourth sequence (C8-Peptide) that could be cleaved extracellularly proximal to sites of COVID-19 infection. Once the DF-COV cargo is cleaved from the MAb, it enters the cell through its ionophore activity, and releases Zn through COVID proteolysis between the two halves of its Zn finger. Addition of an Arg-Lys rich MTS at either the amino or carboxyl terminus of the peptide can facilitate Zn release through targeting the payload into the lysosome.

No broadly effective treatment for advanced SARS-CoV-2 disease has emerged. Multiple anti-COVID-19 MAb cocktails did not achieve efficacy in severe COVID-19 disease trials. A MAb-DFCOV conjugate could reposition MAb cocktails for success through a synergistic attack on COVID, at the point of cell entry provided by the MAb, and at COVID replication provided by its Zn payload. Further through the capacity of intracellular Zn to counteract life-threatening SARS syndromes, such as lost pulmonary surfactant, bacterial pneumonias, and failed antibody maturation, time for the conjugate to work is afforded.

Numerous studies have shown that oral Zn has PEP activity against “colds”, although this PEP activity is incomplete, and has not specifically been serotyped to COVID infections. At least three retrospective clinical studies (Frontera, Derwand, Vogel-Gonzalez) have supported PEP activity for Zn in COVID-19 infection. No study has demonstrated Zn efficacy in COVID-19 PREP. Oral Zn is unsuited for PREP, because the toxicities from taking sustained, high amounts of oral Zn, which feeds extracellular compartments, are many, including profound anemia due to induced copper deficiency, gastrointestinal symptoms, and blood clotting abnormalities. The risk of blood clotting abnormalities in SARS-CoV-2 is exacerbated by extracellular Zn.

A Zn-conjugated, (long-acting) MAb could be a safe and broadly efficacious tool for PREP against COVID 19 infection. The conjugate creates an anti-COVID reservoir of neutralizing antibodies and Zn that is activated only when needed, upon COVID exposure. When the MAb also recognizes conserved COVID-19 determinants, the conjugate will be active as well against emergent variants, which is an important superior characteristic for pandemic control when contrasted to currently-approved COVID vaccines. Such conjugates could extend the efficacy of current vaccines by inhibiting emergence of S antigen variants.

Zn is removed from the intracellular reservoir by multiple COVID-19 proteins with Zn-binding pockets formed in protein maturation. Zn chelation by these proteins creates intracellular Zn deficiency, and an environment permissive to COVID-19 replication. At least five COVID-19 proteins coordinate Zn. COVID-19 polymerase has been crystallized, and its structure has two Zn binding pockets situated in the middle of the protein that facilitate protein folding, away from its active site. The papain-like protease of COVID-19 is structured as a trimer, each unit of which contains a Zn finger. Coronavirus helicases (nsp-13) are all highly conserved; the nsp-13 of MERS, like SARS-CoV-2 a human respiratory pathogen emerging from zoonotic transmission, has been crystallized and found to have three domains each with a Zn-coordinated core. COVID-19 replicase (nsp-14) contains a Zn finger. The COVID-19 heterodimer formed between nsp-10 and nsp-16 chelates Zn. Other proteins unique to COVID-19 whose structures have not yet been resolved could also bind Zn, or could even primarily function to enhance viral replication as Zn chelators. By removing so much intracellular Zn during protein folding, COVID 19 starves the cell of Zn, creating a permissive environment for viral replication. Intracellular Zn treats COVID-19 infection by inhibiting RNA polymerase and COVID proteases, thereby blocking the accumulation of matured COVID proteins.

With increasing COVID-19 replication, more and more matured viral proteins accumulate, sequestering free intracellular Zn, thereby creating an intracellular Zn-deficiency state associated with multiple SARS syndromes. Absent Zn protection, lung surfactant could be damaged by Cd-like toxins released by infection from the pulmonary epithelium, such as occurs in infant pulmonary distress syndrome. Zn deficiency is an accelerant of bacterial pneumonia, particularly in nursing home elderly who are most susceptible to SARS-CoV-2 pneumonias. Normal maturation of B cells to mature plasma cells producing high affinity Ig1/2 antibodies requires a divalent cation-coordinated STAT 3 pre-activation complex that utilizes zinc fingers to homodimerize. Consequently, antibody maturation is blocked even in the presence of toxic levels of Il-6 (“cytokine storms”) produced by the body in its futile effort to drive plasma cell maturation to shut down COVID-19 propagation. Individuals suffering cytokine storm have Zn deficiency compared against other COVID-19 infected individuals.

Zn loaded by COVID-19 proteins during viral replication unbalances Zn homeostasis, depleting intracellular Zn while flooding the extracellular compartment with Zn during viral shedding. A therapeutic for rebalancing extracellular Zn is envisioned as an anti-COVID MAb linked to a vacant Zn finger that can chelate Zn without catalysis, such as the well-characterized HIV capsid Zn fingers, synthetic CRD with 4 coordinating cysteines (4C), 3 cysteines and one histidine (3CH), 2 cysteines and 2 histidines (2C2H), or any other Zn-coordinating peptides. The MAb could be directed against any of the COVID proteins that are expressed on the viral surface. After the MAb lyses virus, the Zn finger mops up excess Zn at extracellular sites before extracellular Zn toxicity can initiate myocardial infarcts, pulmonary emboli, or occlusive strokes. As these “self-loading” Zn chelators lack intrinsic ionophore activity, a membrane translocation sequence could be appended, so trafficking them intracellularly to the lysosome for Zn release. A “release switch” decoupling the Zn “mop” from the MAb could be engineered as any of the sequences cleaved by cellular proteases activated during COVID 19 infection (Furin, TMPRSS2, caspase 8, cathepsin B) also illustrated in SEQ ID 5-12. Additional specificity of these therapeutics could be enhanced through placing an arterial catheter for drug delivery near the site of thrombosis.

A free Zn finger, conjugated to the carboxyl (or amino) terminus of any MAb, could further be used to purify the conjugate over a Zn affinity column, much in the manner that a 6×His or biotin tag currently functions. Unlike these other artificial tags which are not used in GMP manufacture, the Zn finger tag is a natural product that could be cleaved off (or administered) safely. This invention is suitable for GMP manufacture and MAb purification in a simple, cost effective procedure. Extracellular Zn overload is caused when COVID-19 is shed from infected cells, undergoes immune attack, and is degraded. The numerous COVID-19 proteins that have coordinated Zn during COVID replication are broken down, showering this Zn into the bloodstream. Released Zn activates a cascade of clotting factors, resulting in thrombosis. Platelets, the primary reservoir for circulating zinc, play into this coagulopathy because hyperzincemia directly activates platelets, which in turn could shed their zinc in a futile attempt to restore intracellular zinc homeostasis. Coagulopathy marks COVID-19 progression, and is a lead mortality factor in SARS. Anosmia, an early symptom of COVID-19 infection in otherwise healthy, young individuals, is another indicator of extracellular zinc overload. While severe Zn deficiency can also cause anosmia, its onset in COVID-19 infection appears much later at 10-14 days in association with SARS symptoms, often in individuals with other risk factors for Zn deficiency.

Healthy, young individuals infected with COVID-19 suffer heart attacks, sudden deaths most likely from pulmonary emboli, and other thrombotic events. Further, these young otherwise healthy individuals suffer thrombotic strokes, a pathology that in normal times is confined to the elderly, from which persistent physical and mental abnormalities ensue. Such thrombotic events are consistent with an extracellular “Zn storm,” in analogy to the hypercompensatory “cytokine storm” in SARS-CoV-2. Extracellular Zn overload is exacerbated when healthy individuals self-medicate with oral Zn supplements to toxic levels.

A critical feature of these DF-COV derivatives is to rebalance Zn homeostasis in COVID-19 infection. Specifically, these Zn porting peptides replenish intracellular Zn reservoirs, shut off COVID 19 replication, and thereby end the export of Zn coordinated to COVID 19 proteins. In contrast, oral or intravenous free Zn immediately contributes to systematic overload with associated toxicities, and only as a byproduct can act intracellularly. Peptides with a vacant Zn coordination site are further made to specifically absorb excess extracellular Zn.

Influenza epidemics have multiple parallels to SARS-CoV-2. They are associated with deaths from bacterial pneumonia, particularly in the elderly. Pandemic influenza infections in 1918 came in three waves, as each wave failed to induce durable antibody protection. Importantly, influenza RNA polymerase, like COVID RNA polymerase, is potently inhabitable by Zn. A synthetically-evolved anti-influenza MAb-Zn conjugate carrying a Zn payload released by an influenza-activated proteolysis could specifically target influenza replication. An anti-influenza MAb recognizing a conserved influenza antigen, conjugated to a Zn porter through a linker copying the hemaglutinin cleavage sequence, could become the long-sought after universal influenza preventive. Moreover, the MAb-Zn conjugate could be optimized through a linker specific to the strain of influenza circulating during any influenza season (SEQ ID 15-18). Intracellular release of Zn can be designed by trafficking the Zn porter to the lysosome through a membrane translocation sequence. Since influenza uses a caspase 3 apoptosis pathway, intracellular Zn release can alternatively be engineered into the peptide through insertion of a caspase 3 recognition sequence (DEVD) between the two halves of the Zn finger. SEQ ID 14 demonstrates such a construct designed with tandem caspase 3 recognition sequences so as to preserve spacing CRDs. Such MAb-Zn conjugates are useful insurance against the emergence of a future influenza pandemic.

Zn deficiency is thought to contribute to immune suppression in cancer, so that linking Zn porters to an anti-cancer MAb ADC, particularly MAb targeted against cancer neo-antigen, creates a novel oncoimmunologic derivative. Such an anti-cancer ADC could carry two payloads, firstly anti-cancer drug, and secondly Zn in tandem. The anti-cancer toxin could be targeted to directly kill cancer cells, as in conventional ADC. The Zn payload could re-invigorate the anti-cancer response to neoantigen, by uptake into a dendritic cell, endocytosis into an antigen presenting cell, or through Fc-related immune activation. Ionophore activity of the Zn porter also efficiently carries the toxin into the cell.

Synthetically-evolved MAb-Zn conjugates specific for the release of Zn at sites of cancer invasion can be created via a linker composed of a cleavage sequence recognized in matrix proteolysis. As one example, matrix metalloproteinases are upregulated by many invasive cancers in a cancer-type specific manner. Since tissue invasion is the cause of death in cancer, this derivative delivers Zn payload to the site where it is most needed. The genetic sequence of both the Zn payload linker, and the cancer MAb, are engineered specifically for each cancer, or type of cancer. Zinc is then released intracellularly via uptake by antigen presenting cells, or when trafficked to the lysosome when attached to a membrane translocation sequence.

### EXEMPLARY DESCRIPTION OF THE INVENTION

Sequences indicated are shown only by way of example representing any sequence, or derivative thereof, with the specified parameters.

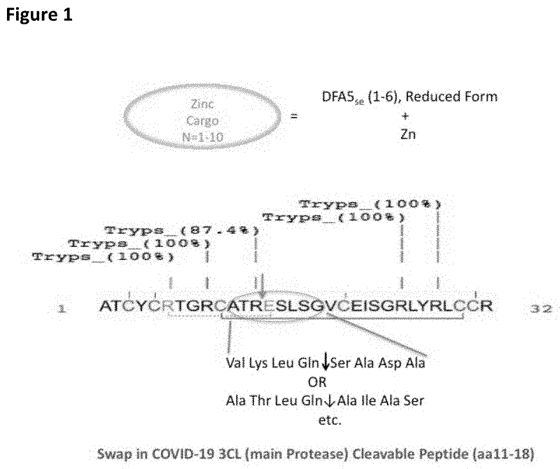

As shown in FIG. 1, Human Alpha Defensins are a conserved family of peptides with six cysteines, that when reduced structure a Zn finger that chelates Zn. Alpha defensin 5 (ADF5, SEQ ID No. 13) is a gut peptide that in its oxidized form links Cys 10 to Cys 30, folding the peptide into an open ribbon that exposes 5 perfect trypsin cleavage sites. Because trypsin is an abundant protease in the gut, oxidized alpha defensin 5 is rapidly degraded. In contrast, Zn chelation to the reduced from of alpha defensin 5 leaves accessible only an imperfect (87%) trypsin site in the span between the Cys-rich sequences, rendering alpha defensin 5 a stable carrier of ionic Zn++. Trypsin cleavage at the imperfect site (aa 13) separates the Zn finger, dissociating Zn. As trypsin is not present in the lung or other sites of COVID-19 infection, discharge at sites of active COVID-19 infection would not be favored. This invention creates a synthetically evolved defensin (DFAₛₑ) with either a COVID-19 3Cl (main) protease cleavage site genetically engineered (aa11-18, or 12-19) to replace the imperfect trypsin site (DF-MCP, SEQ ID NO. 1); or a SARS PLP (DF-PLP, SEQ ID NO. 2) cleavage site (not illustrated, eg. Glu Leu Asn Gly Gly↓Ala Val Thr Arg). Proteolytic cleavage at the engineered site releases Zn in a manner controlled by the level of COVID-19 infection. Replacement of the natural trypsin spacer by COVID-19 cleavage sites in the synthetic defensin is engineered to maintain the same molecular spacing between the cysteines, although peptides with 1, 2, 3, or more sites in tandem are also possible. The Synthetic Defensin, containing the COVID-19 Zn release function, can be administered alone, or piggybacked to increase Zn cargo (1-10 moieties, or more).

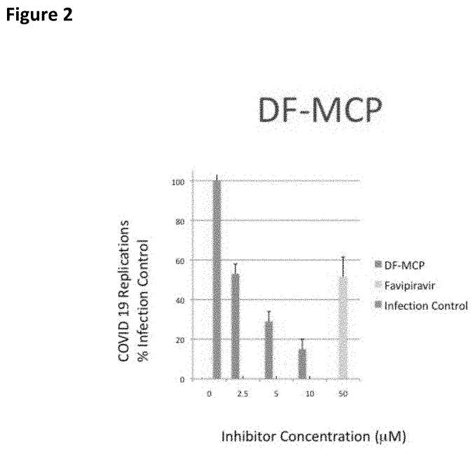

As shown in FIG. 2, adherent cells were seeded at 3×10\hat{ }5 cells/well of a 12 well plate on d1. d2—cells were infected with COVID 19 virus MOI of 0.01 in the presence of 1× compound or carrier control in a total volume of 250 μl, incubated 1 hour @ 37 degrees C./5% CO2 on rocker. Plates were washed, complete media/1× compound added to a total volume of 500 μl, and plates were incubated @ 37 degrees C./5% CO2. Plates were harvested after 72 hours infection on d5 at a time when CPE is pronounced in infection controls, and COVID infection quantitated by qPCR. Infection control (carrier, 0 μM compound); DF-MCP (SEQ ID NO. 1), human alpha defensin 5 synthetically-evolved to contain a 3CL-major covid protease cleavage site inserted between the two halves of the defensin Zn finger (2.5, 5 and 10 μM Zn-coordinated peptide); favipiravir (50 μM). Readout is % COVID 19 replication of trial compound/infection control. All samples were done in triplicate, and qPCRs performed in triplicates. The result is representative of three assays.

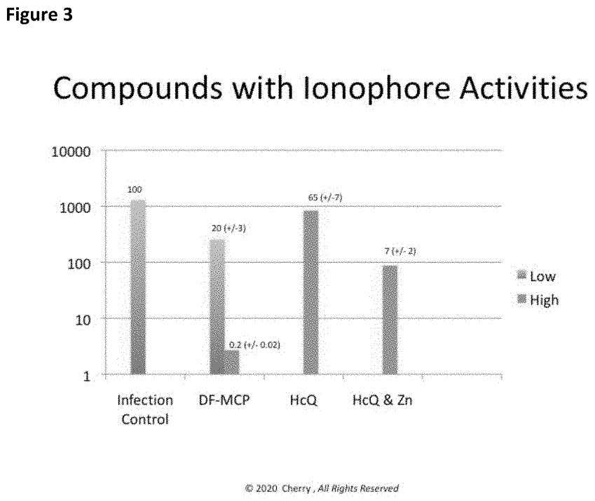

FIG. 3 represents a graph showing the potency of Zn in combination with ionophore and compounds with ionophore activity in inhibiting COVID-19 replication. Adherent cells were seeded at 3×10\hat{ }5 cells/well of a 12 well plate on d1. d2—cells were infected with COVID 19 virus MOI of 0.01 in the presence of 1× compound or carrier control in a total volume of 250 μl, and incubated 1 hour @ 37 degrees C./5% CO2 on rocker. After plates were washed, complete media/1× compound was added to a total volume of 500 μl, and were incubated @ 37 degrees C./5% CO2. Plates were harvested after 72 hours infection on d5 at a time when CPE is pronounced in infection controls, and COVID infection quantitated by qPCR. Infection control (carrier, 0 μM compound); DF-CMP, derivatized, Zn-loaded alpha defensin 5 (SEQ ID NO. 1), which has intrinsic ionophore activity (5 μM, 20 μM); HcQ (hydroxychloroquine, 50 μM), HCQ (50 μM) & Zn (Zn, 2 μM). Readout is relative qPCR units, while the number directly above each bar is the replication ratio (trial compound/infection control (%)). All samples were done in triplicate, and qPCRs performed in triplicates.

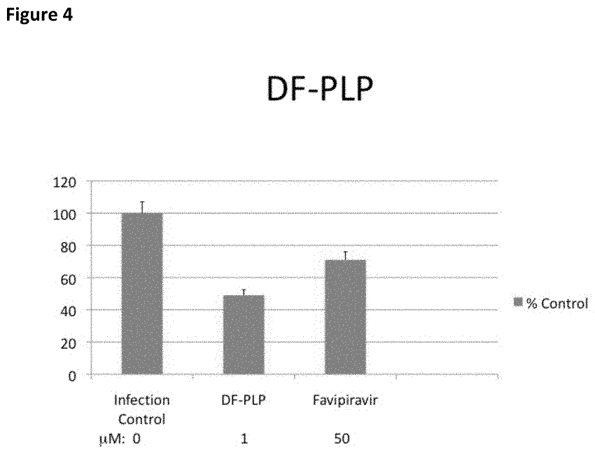

FIG. 4 depicts anti Covid-19 Activity of DF-PLP (SEQ ID NO. 2). Adherent cells were seeded at 3×10\hat{ }5 cells/well of a 12 well plate on d1. d2—cells were infected with COVID 19 virus MOI of 0.01 in the presence of 1× compound or carrier control in a total volume of 250 μl, and incubated 1 hour @ 37 degrees C./5% CO2 on rocker. After plates were washed, complete media/1× compound was added to a total volume of 500 μl, and were incubated @ 37 degrees C./5% CO2. Plates were harvested after 72 hours infection on d5 at a time when CPE is pronounced in infection controls, and COVID infection quantitated by qPCR. Infection Control (carrier, 0 μM) DF-PLP (SEQ ID NO. 2), alpha defensin 5 synthetically evolved to contain a COVID-19 pepsin-like protease cleavage site between the two halves of its Zn finger (1 μM peptide, Zn loaded); Favipriravir (50 μM). Readout is % COVID 19-replication of compound treated samples relative to untreated infection control. All samples were done in triplicate, and qPCRs performed in triplicates.

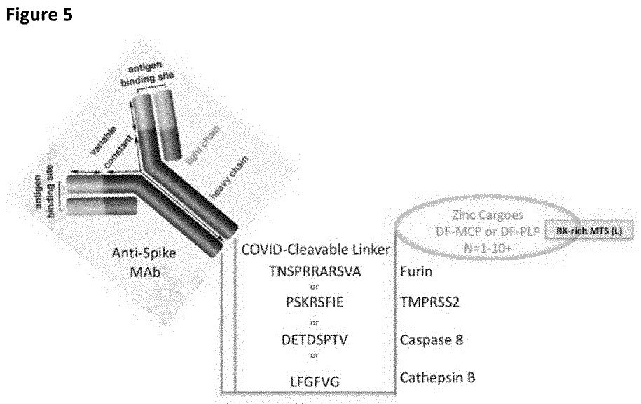

FIG. 5 represents a schematic of a Zn Payload precisely-targeted through coupling a COVID-specific MAb to a COVID-activated Zn Porter/ionophore. Genetic construct of the MAb-Zn conjugate encodes a 5′heavy chain MAb recognizing COVID-19 spike protein, or other external protein, linked to a 3′ Zn porter through a sequence that is cleavable by extracellular proteases active at sites of COVID 19 infection. Release sequences include Furin (SEQ ID NO. 5 and SEQ ID NO. 6), TMPRSS2 (SEQ ID NO. 7 and SEQ ID NO. 8) Cathepsin B (SEQ ID NO. 9 and SEQ ID NO. 10), or Caspase 8 ((SEQ ID NO. 11 and SEQ ID NO. 12) cleavage sites. Addition 3′ of an RK-rich membrane translocation sequence (MTS) is designed to facilitate cell penetration and intracellular trafficking to the lysosome for enhancement of Zn release (SEQ ID NO. 3 and SEQ ID NO. 4). Multiple Zn cargoes can be loaded onto one heavy chain in tandem reducing the amount of MAb required for efficacy of the conjugate drug. Co-transfectants expressing the cargo-carrying heavy chain conjugate and light chain are spontaneously assembled in a hybridoma to produce the complete MAb-conjugate therapeutic.

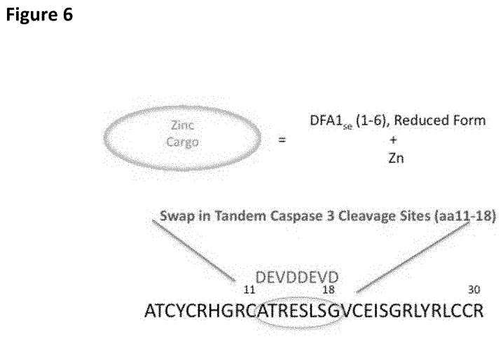

FIG. 6 shows a diagram of a synthetically-evolved alpha Defensin targeted to release Zn inside of influenza-infected cells. Influenza type A induces caspase-3, thereby triggering apoptosis and viral shedding. Two caspase-3 recognition sites (DEVD) are inserted in tandem replacement of amino acids 11-18 (SEQ. ID No. 14) of alpha defensin 5, thereby conserving the spacing between the two halves of the CRD, although any number of caspase 3 can be inserted in tandem in order to optimize targeted release. The synthetic defensin can be administered alone, or linked in multiples of 1-10, or even more, or onto another agent, such as an anti-influenza MAb. Since caspase-3 induction is a later event in the course of cellular infection, an alternative design that can inhibit influenza replication immediately engineers a membrane translocation sequence (L, see Sequence ID NO. 15) onto the synthetic defensin. The MTS traffics the Zn cargo into the lysosome, where it is released by a combination of proteolysis and acidification. Another preferred realization of this invention is a genetic construct that through gene therapy can be engineered into a patient's own neutrophils, that would then chemotax to the sites of active infection.

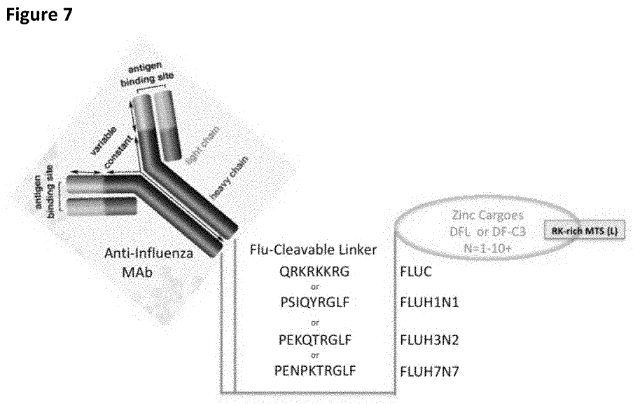

FIG. 7 shows a schematic of a Precisely targeted Zn Payload through coupling an Influenza-specific MAb to a Zn Porter/ionophore. Genetic construct encoding 5′ heavy chain MAb, linked to its zinc cargo through a hemaglutinin (HA) protease site specific to each circulating influenza strain (SEQ ID NO. 15 through SEQ ID NO. 18), that through its cleavage releases Zn cargo that is moved intracellularly due to ionophore activity of the Zn peptide (DF). Addition 3′ of an RK-rich membrane translocation sequence (L) is designed to facilitate cell penetration and Zn release in the lysosome (L, SEQ ID NO. 15 and SEQ ID NO. 18). Multiple Zn cargoes can be loaded onto one heavy chain in tandem reducing the amount of MAb required for the conjugate drug to achieve therapeutic threshold. Co-transfectants expressing the cargo-carrying heavy chain conjugate and light chain are spontaneously assembled by the Hybridoma.

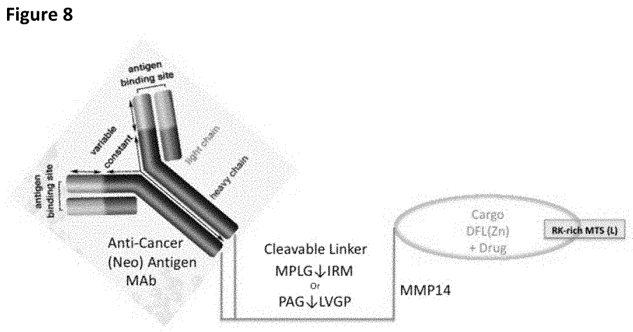

FIG. 8 shows a schematic of a Zn Payload Precisely targeted to a cancer through coupling to a MAb specific to a cancer (Neo) Antigen. Genetic construct encoding 5′ heavy chain MAb, a linker that is cleavable by extracellular proteases active at sites of cancer invasion, such as matrix metalloproteinase 14 (MMP14) in breast cancer; a synthetically-evolved zinc porter (DFL-Zn), that moves zinc cargo intracellularly due to ionophore activity, then releases free zinc upon entering the lysosome (L) using a 3′ RK-rich membrane translocation sequence. Multiple zinc cargoes and/or an anti-cancer chemotherapeutic can be loaded onto one heavy chain in tandem, so that the chemotherapeutic takes advantage of the ionophore to penetrate the cell. The conjugate drug enhances the therapeutic efficacy of its components. Co-transfectants expressing the cargo-carrying heavy chain conjugate and light chain are spontaneously assembled by the Hybridoma.

Although the present invention has been described with reference to specific embodiments, workers skilled in the art will recognize that many variations may be made therefrom, for example in the particular experimental conditions herein described, and it is to be understood and appreciated that the disclosures in accordance with the invention show only some preferred embodiments and objects and advantages of the invention without departing from the broader scope and spirit of the invention. It is to be understood and appreciated that these discoveries in accordance with this invention are only those which are illustrated of the many additional potential applications that may be envisioned by one of ordinary skill in the art, and thus are not in any way intended to be limiting of the invention. Accordingly, other objects and advantages of the invention will be apparent to those skilled in the art from the detailed description together with the claims.

I claim:

1. A composition for use in an anti-viral therapeutic method in a subject having a viral infection comprising:
a. conjugating a monoclonal antibody (Mab) directed to an antigen site of viral infection to a Zn-porting ionophore carrying a Zn cargo;
b. targeting delivery by administration of a sufficient amount of the Zn cargo at the cellular site of the viral infection presenting an antigen; and
c. intracellularly releasing Zn,
wherein the MAb and Zn provide a synergistic activity to the viral infection of the subject.

2. The composition of claim 1 wherein the viral infection is COVID-19.

3. The composition of claim 1 wherein the viral infections are multiple SARS syndromes and a sequelae of SARS-CoV-2 disease.

4. The composition of claim 3 wherein the SARS syndrome is lost pulmonary surfactant, bacterial pneumonias or failed antibody maturation.

5. The composition of claim 3 wherein the disease is a thrombotic event, or infant pulmonary distress syndrome.

6. The composition of claim 1 wherein the MAb includes MAb drugs directed at COVID-19 external antigens.

7. The composition of claim 1 where the MAb is specific for Spike (S) protein.

8. The composition of claim 1 where the MAb is linked to the Zn porter by a genetic sequence containing a Furin-cleavage site (FU) encoded by COVID 19, or any other furin-cleavage site.

9. The composition of claim 1 where the MAb is linked to the Zn porter by a sequence encoding a TMPRSS2 cleavage site.

10. The composition of claim 1 where the MAb is inked to the Zn porter by a sequence containing a Caspase 8 cleavage site (C8).

11. The composition of claim 1 where the MAb is linked to the Zn porter by a sequence containing a cathepsin B cleavage site (CB).

12. The composition of claim 1 where the Zn porter is a peptide having an Arg-Lys Rich MTS at the amino or carboxyl terminus.

13. The composition of claim 1 where the Zn-porting ionophore is alpha defensin 5.

14. The composition of claim 1 where the Zn porter is selected from the group consisting of DFAse, SEQ ID No. 1, SEQ ID No. 2, SEQ ID No. 3, SEQ ID No. 4, SEQ ID No. 13, SEQ ID No. 14, and SEQ ID No. 21.

15. The composition of claim 1 where the composition promotes the efficacy and extends the duration of protection of Post-Exposure Prophylaxis (PEP), Pre-Exposure Prophylaxis (PREP), or treatment of COVID-19 infections.

16. The composition for use in a method of claim 1 wherein said composition further comprises an adjuvant optionally wherein said adjuvant is selected from a group consisting of complete Freund's adjuvant, incomplete Freund's adjuvant, Montanide ISA-51, LAG-3, aluminum phosphate, aluminum hydroxide, alum, and saponin.

17. A composition of claim 1 for the prevention or treatment of influenza, including pandemic influenza, through the specific modification of MAb and cleavage sequences to render the therapeutic specific to influenza.

18. A composition of claim 17 where the modified DFₛₑ zinc porter is SEQ ID No. 15.

19. A composition of claim 17 where the specific modifications to MAb and cleavage sequences render the therapeutic specific to the type of influenza circulating during any given season.

20. A composition of claim 17 where the sequences linked to the MAb are selected from SEQ ID No. 16, SEQ ID No. 17, SEQ ID No. 18, SEQ ID No. 19.

21. A composition for use in an anti-cancer therapeutic method in a subject having cancer comprising:
a. conjugating a monoclonal antibody (MAb) directed to an antigen site of a cancer cell to a Zn-porting ionophore carrying Zn cargo;
b. targeting delivery by administration of a sufficient amount of the Zn cargo at the cellular site of the cancer cell; and
c. intracellularly releasing Zn,
wherein a synergistic activity against the cancer is attained.

22. The composition of claim 21 wherein the MAb is directed to a cancer neo-antigen.

23. The composition of claim 21 where the Zn porter is a defensin.

24. The composition of claim 21 where Zn porter is a synthetic defensin.

25. The composition of claim 21 where the Zn porter is SEQ ID No. 14.

26. The composition of claim 21, where the MAb is connected to the Zn-porting ionophore via a linker cleavable by proteases active during cancer tissue invasion.

27. The composition of claim 21 where the linker is cleavable by MMP proteases.

28. The composition of claim 21, where the linked sequence is SEQ ID No. 20, containing a proteolytic site for MMP-14 that is activated during tissue invasion by breast and other cancers.


SEQUENCE LISTING (21 sequences)

SEQ ID NO: 1 - protein, 32 aa, Homo sapiens
ATCYCRHGRCVKLQSADAVCEISGRLYRLCCR

SEQ ID NO: 2 - protein, 33 aa, Homo sapiens
ATCYCRHGRCELNGGAVTRVCEISGRLYRLCCR

SEQ ID NO: 3 - protein, 41 aa, Homo sapiens
ATCYCRHGRCVKLQSADAVCEISGRLYRLCCRRKKRRQRRR

SEQ ID NO: 4 - protein, 42 aa, Homo sapiens
ATCYCRHGRCELNGGAVTRVCEISGRLYRLCCRRKKRRQRRR

SEQ ID NO: 5 - protein, 43 aa, Homo sapiens
TNSPRRARSVAATCYCRHGRCVKLQSADAVCEISGRLYRLCCR

SEQ ID NO: 6 - protein, 44 aa, Homo sapiens
TNSPRRARSVAATCYCRHGRCELNGGAVTRVCEISGRLYRLCCR

SEQ ID NO: 7 - protein, 40 aa, Homo sapiens
PSKRSFIEATCYCRHGRCVKLQSADAVCEISGRLYRLCCR

SEQ ID NO: 8 - protein, 41 aa, Homo sapiens
PSKRSFIEATCYCRHGRCELNGGAVTRVCEISGRLYRLCCR

SEQ ID NO: 9 - protein, 38 aa, Homo sapiens
LFGFVGATCYCRHGRCVKLQSADAVCEISGRLYRLCCR

SEQ ID NO: 10 - protein, 39 aa, Homo sapiens
LFGFVGATCYCRHGRCELNGGAVTRVCEISGRLYRLCCR

SEQ ID NO: 11 - protein, 40 aa, Homo sapiens
DETDSPTVATCYCRHGRCVKLQSADAVCEISGRLYRLCCR

SEQ ID NO: 12 - protein, 41 aa, Homo sapiens
DETDSPTVATCYCRHGRCELNGGAVTRVCEISGRLYRLCCR

SEQ ID NO: 13 - protein, 32 aa, Homo sapiens
ATCYCRTGRCATRESLSGVCEISGRLYRLCCR

SEQ ID NO: 14 - protein, 41 aa, Homo sapiens
ATCYCRTGRCATRESLSGVCEISGRLYRLCCRRKKRRQRRR

SEQ ID NO: 15 - protein, 32 aa, Homo sapiens
ATCYCRTGRCDEVDDEVDVCEISGRLYRLCCR

SEQ ID NO: 16 - protein, 49 aa, Homo sapiens
QRKRKKRGATCYCRTGRCATRESLSGVCEISGRLYRLCCRRKKRRQRRR

SEQ ID NO: 17 - protein, 50 aa, Homo sapiens
PSIQYRGLFATCYCRTGRCATRESLSGVCEISGRLYRLCCRRKKRRQRRR

SEQ ID NO: 18 - protein, 50 aa, Homo sapiens
PEKQTRGLFATCYCRTGRCATRESLSGVCEISGRLYRLCCRRKKRRQRRR

SEQ ID NO: 19 - protein, 51 aa, Homo sapiens
PENPKTRGLFATCYCRTGRCATRESLSGVCEISGRLYRLCCRRKKRRQRRR

SEQ ID NO: 20 - protein, 48 aa, Homo sapiens
MPLGIRMATCYCRTGRCATRESLSGVCEISGRLYRLCCRRKKRRQRRR

SEQ ID NO: 21 - protein, 30 aa, Homo sapiens
ACYCRIPACIAGERRYGTCIYQGRLWAFCC